(19) United States
(12) Patent Application Publication
(10) Pub. No.: US 2025/0087337 A1
(43) Pub. Date: Mar. 13, 2025

(54) LARGE LANGUAGE MODEL-BASED TRANSLATOR FOR MEDICAL IMAGING METADATA

(71) Applicant: Siemens Medical Solutions USA, Inc., Malvern, PA (US)

(72) Inventors: Alexander Hans Vija, Evanston, IL (US); Francesc dAssis Massanes Basi, Chicago, IL (US)

(21) Appl. No.: 18/631,152

(22) Filed: Apr. 10, 2024

Related U.S. Application Data
(60) Provisional application No. 63/581,127, filed on Sep. 7, 2023.

(51) Int. Cl. G16H 30/40; G16H 30/20

(52) U.S. Cl. CPC G16H 30/40; G16H 30/20

(57) ABSTRACT

Systems and methods for converting medical imaging metadata from a first format to a second format are provided. Medical imaging metadata in a first format and instructions are received. The medical imaging metadata is converted from the first format to a second format based on the instructions using a machine learning based model. The medical imaging metadata in the second format is output.

### CROSS-REFERENCE TO RELATED APPLICATIONS

This application claims the benefit of U.S. Provisional Application No. 63/581,127, filed Sep. 7, 2023, the disclosure of which is herein incorporated by reference in its entirety.

### TECHNICAL FIELD

The present invention relates generally to a translator for medical imaging metadata, and in particular to an LLM (large language model)-based translator for medical image metadata.

### BACKGROUND

Medical imaging typically comprises imaging data (e.g., pixel or voxel data) and metadata. Such medical imaging metadata is often embedded in an image header in a DICOM (digital imaging and communications in medicine) format. Adopting the latest DICOM format can be costly and is typically met with reluctance, as different vendors use slightly varied implementations of DICOM since private tags are permitted under the DICOM format standard. Additionally, the DICOM format standard often changes and vendors must adapt to the updated standard to remain compliant. As a result, medical imaging metadata received from various source systems in different formats must be converted to a single format for analysis. Conventionally, conversion of medical imaging metadata between formats is performed by hard coding the conversion for each of the formats. However, such conventional conversion of medical imaging metadata is time-consuming and expensive.

### BRIEF SUMMARY OF THE INVENTION

In accordance with one or more embodiments, systems and methods for converting medical imaging metadata from a first format to a second format are provided. Medical imaging metadata in a first format and instructions are received. The medical imaging metadata is converted from the first format to a second format based on the instructions using a machine learning based model. The medical imaging metadata in the second format is output.

In one embodiment, the machine learning based model is an LLM (large language model). One or more prompts comprising 1) the medical imaging metadata in the first format and 2) the instructions are received.

In one embodiment, the machine learning based model receives as input the medical imaging metadata in the first format and the instructions and generates as output the medical imaging metadata in the second format.

In one embodiment, the medical imaging metadata is metadata associated with an acquisition of one or more medical images of a patient. The medical imaging metadata comprises patient health information of the patient and image acquisition parameters of the one or more medical images.

In one embodiment, the first format and the second format are different implementations of a DICOM (digital imaging and communications in medicine) format.

In one embodiment, the instructions comprise instructions for converting the medical imaging metadata from the first format to the second format.

In one embodiment, the medical imaging metadata is converted from the first format to the second format in response to determining that the medical imaging metadata in the second format includes or is missing information for a class of patients that would influence a future processing step.

In one embodiment, information is extracted from a medica image associated with the medical imaging data using a machine learning based image assessment model. The extracted information is compared with the medical imaging data to confirm accuracy of the medical imaging data.

These and other advantages of the invention will be apparent to those of ordinary skill in the art by reference to the following detailed description and the accompanying drawings.

### BRIEF DESCRIPTION OF THE DRAWINGS

FIG. 1 shows a method for converting medical imaging metadata from a first format to a second format using a machine learning based model, in accordance with one or more embodiments;

FIG. 2 shows a workflow for converting medical imaging metadata from a first format to a second format using an LLM, in accordance with one or more embodiments;

FIG. 3 shows an exemplary artificial neural network that may be used to implement one or more embodiments;

FIG. 4 shows a convolutional neural network that may be used to implement one or more embodiments;

FIG. 5 shows a schematic structure of a recurrent machine learning model that may be used to implement one or more embodiments; and

FIG. 6 shows a high-level block diagram of a computer that may be used to implement one or more embodiments.

### DETAILED DESCRIPTION

The present invention generally relates to methods and systems for an LLM (large language model)-based translator for medical imaging metadata. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for an LLM-based translator for translating medical imaging metadata between various formats. Advantageously, the LLM-based translator enables conversion of medical imaging metadata from a first format associated with a vendor/model/version/release to a second format associated with any other vendor/model/version/release, thereby facilitating data communication and essentially eliminating the need for a medical imaging metadata format standard.

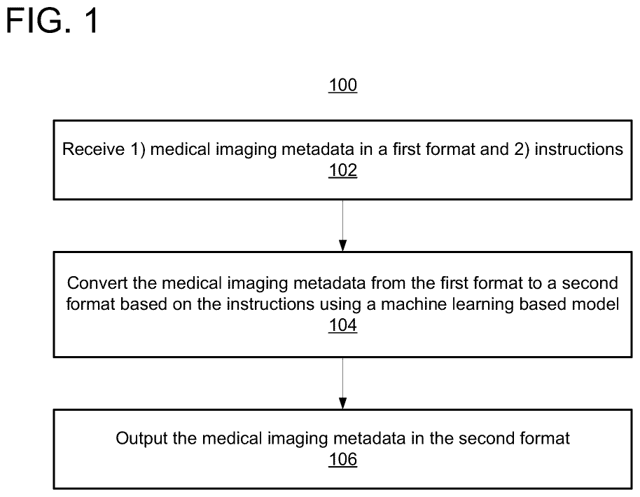

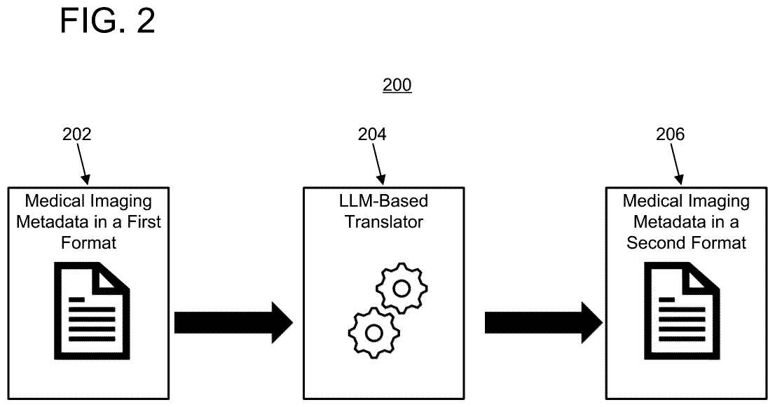

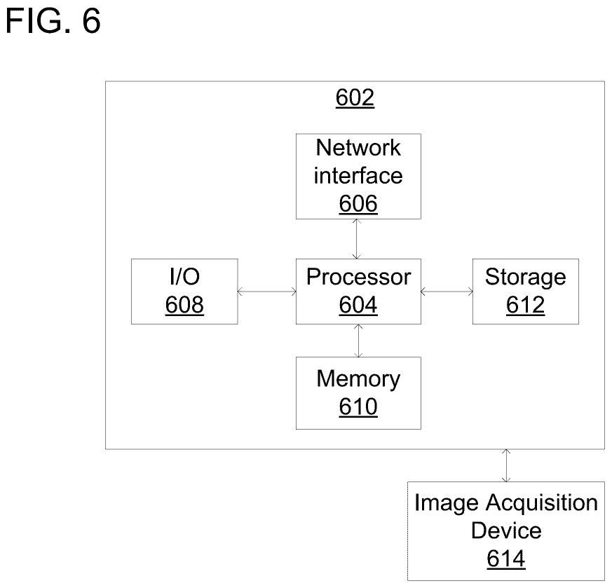

FIG. 1 shows a method 100 for converting medical imaging metadata from a first format to a second format using a machine learning based model, in accordance with one or more embodiments. The steps of method 100 may be performed by one or more suitable computing devices, such as, e.g., computer 602 of FIG. 6. FIG. 2 shows a workflow 200 for converting medical imaging metadata from a first format to a second format using an LLM, in accordance with one or more embodiments. FIG. 1 and FIG. 2 will be described together.

At step 102 of FIG. 1, 1) medical imaging metadata in a first format and 2) instructions are received. In one example, as shown in workflow 200 of FIG. 2, the medical imaging metadata in the first format is medical imaging metadata 202 in a first format.

The medical imaging metadata may be any metadata associated with the acquisition of one or more medical images of a patient. For example, the medical imaging metadata may comprise patient health information (such as, e.g., patient name, date of birth, etc.) of the patient and image acquisition parameters (such as, e.g., image dimensions, voxel size, repetition time, voxel data type, etc.) of the one or more medical images. In one embodiment, the first format of the medical imaging metadata is a DICOM format. However, the first format may be any other suitable standard or non-standard format (e.g., Interfile or a proprietary format).

The one or more medical images, associated with the medical imaging metadata, may be of any suitable modality or modalities, such as, e.g., CT (computed tomography), MRI (magnetic resonance imaging), US (ultrasound), x-ray, or any other medical imaging modality or combinations of medical imaging modalities, and may comprise 2D (two dimensional) images and/or 3D (three dimensional) volumes. The medical imaging metadata may be generated by an image acquisition device by which the one or more medical images are acquired.

The instructions may be any suitable instructions for converting the medical imaging metadata from the first format to a second format. An instruction refers to guidelines or directions provided to guide the behavior and output of the machine learning based model. An instruction may include commands, questions, constraints, requirements, or any other guideline or direction guiding the behavior and output of the machine learning based model. The instructions may include any other information for converting the medical imaging metadata from the first format to a second format, such as, e.g., contextual data. In one example, the instructions are as follows: “Convert the following medical image metadata for system X.”

The medical imaging metadata in the first format and the instructions may be received, for example, from a user interacting with a computer system (e.g., computer 602 of FIG. 6), by loading the medical imaging metadata in the first format and the instructions from a storage or memory of a computer system (e.g., storage 612 or memory 610 of computer 602 of FIG. 6), or by receiving the medical imaging metadata in the first format and the instructions from a remote computer system (e.g., computer 602 of FIG. 6).

At step 104 of FIG. 1, the medical imaging metadata is converted from the first format to a second format based on the instructions using a machine learning based model. In one embodiment, the machine learning based model is an LLM. However, the machine learning based model may be implemented according to any other suitable language model or according to any other suitable machine learning based architecture. In one example, as shown in workflow 200 of FIG. 2, the LLM is LLM-based translator 204 that receives as input medical imaging metadata 202 in the first format (as well as instructions) and generates as output medical imaging metadata 206 in the second format.

The LLM receives as input the medical imaging metadata in the first format and the instructions and generates as output the medical imaging metadata in the second format. The second format may be suitable standard or non-standard format different from the first format. For example, the first and second formats may be different implementations of the DICOM format (e.g., different vendors, models, versions, or releases of the DICOM format). The LLM may receive the medical imaging metadata in the first format and the instructions via one or more prompts. A prompt refers to the input to an LLM for generating a response.

The LLM may be any suitable pre-trained deep learning based LLM. For example, the LLM may be based on the transformer architecture, which uses a self-attention mechanism to capture long-range dependencies in text. One example of a transformer-based architecture is GPT (generative pre-training transformer), which has a multilayer transformer decoder architecture that may be pretrained to optimize the next token prediction task and then fine-tuned with labelled data for various downstream tasks. GPT-based LLMs may be trained using reinforcement learning with human feedback for performing various natural language processing tasks. Other exemplary transformer-based architectures include BLOOM (BigScience Large Open-science Open-access Multilingual Language Model) and BERT (Bidirectional Encoder Representations from Transformers). In one embodiment, the LLM is fine-tuned for the use case of converting the medical imaging metadata from the first format to the second format. The pre-training and fine-tuning are performed during a prior offline or training stage or stages. Once trained, the LLM is applied during an online or inference stage, e.g., to perform step 104 of FIG. 1.

At step 106 of FIG. 1, the medical imaging metadata in the second format is output. For example, the medical imaging metadata in the second format can be output by displaying the medical imaging metadata in the second format on a display device of a computer system (e.g., computer 602 of FIG. 6), storing the medical imaging metadata in the second format on a memory or storage of a computer system (e.g., memory 610 or storage 612 of computer 602 of FIG. 6), or by transmitting the medical imaging metadata in the second format to a remote computer system (e.g., computer 602 of FIG. 6).

Advantageously, embodiments described herein provide for flexibility and allow for quick adaption and interchange of data. Embodiments described herein enable products and systems to change formats (e.g., DICOM formats) as needed, without having to maintain backward compatibility or develop large software packages to accept multiple formats. The (e.g., DICOM) format standard can be replaced using the LLM-based translator in accordance with embodiments described herein.

In one embodiment, method 100 of FIG. 1 may be adapted for converting patient data from a first format to a second format. In this embodiment, at step 102, instead of medical imaging metadata, patient data in a first format is received along with the instructions. At step 104, the patient data is converted from the first format to a second format based on the instructions using a machine learning based model. The first and second formats may be formats associated with different patient databases, such as, e.g., EHRs (electronic health records), EMRs (electronic medical records), PHRs (personal health records), HIS (health information systems), RIS (radiology information systems), PACS (picture archiving and communication systems), LIMS (laboratory information management systems), or any other database or system suitable for storing patient data. At step 106, the patient data in the second format is output. This embodiment allows for mining and augmentation of patient data across various patent databases.

In one embodiment, method 100 of FIG. 1 may be implemented with anti-bias and error checking, analogous to a checksum. In this embodiment, the machine learning based model (e.g., the LLM) is trained to prevent or guard against abuse by biasing when certain information is used for, e.g., a class of patients (or other users) to add, remove, change, or otherwise influence a next processing step resulting in a harm or benefit. For example, the machine learning based model may convert the medical imaging metadata in the first format to the second format in response to determining that the medical imaging metadata in the second format includes or is missing specific information for a class of patients, which, if included or missing, could influence a future processing step.

In one embodiment, a machine learning based image assessment model may be used to assess and extract information from the DICOM file (comprising the medical imaging metadata and an associated medical image) to guard against bias and to confirm/check that the medical imaging metadata is accurate. As the machine learning based model (e.g., the LLM) converts the medical imaging metadata (at step 104 of FIG. 1), the image assessment model may extract information from the medical image to check that the medical imaging metadata is consistent with the information extracted from the medical image. For example, the image assessment model may determine that the sex of the patient as depicted in the medical image is not consistent with the sex of the patient as assigned in the medical imaging metadata and present a warning to the user for confirmation or correction.

In one embodiment, a checksum may be run before and after the conversion of the medical imaging metadata (according to method 100 of FIG. 1) to verify that the payload of the DICOM file (e.g., the medical image) has not been tampered with. In one embodiment, every conversion (according to method 100 of FIG. 1) of medical imaging metadata is logged onto a blockchain.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the machine learning based model utilized at step 104 of FIG. 1, LLM-based translator 204 of FIG. 2, and the machine learning based image assessment model, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

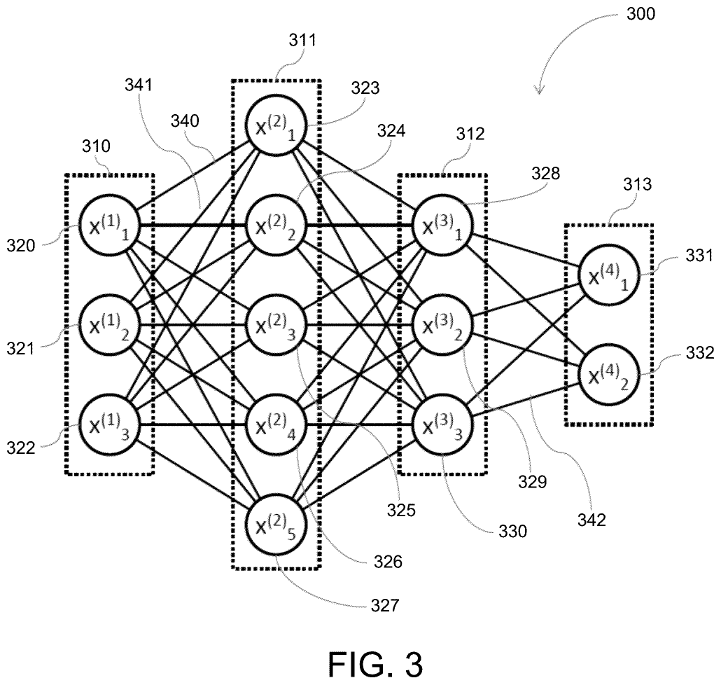

FIG. 3 shows an embodiment of an artificial neural network 300 that may be used to implement one or more machine learning models described herein. Alternative terms for “artificial neural network” are “neural network”, “artificial neural net” or “neural net”.

The artificial neural network 300 comprises nodes 320, . . . , 332 and edges 340, . . . , 342, wherein each edge 340, . . . , 342 is a directed connection from a first node 320, . . . . . . , 332 to a second node 320, . . . , 332. In general, the first node 320, . . . , 332 and the second node 320, . . . 332 are different nodes 320, . . . , 332, it is also possible that the first node 320, . . . 332 and the second node 320, . . . , 332 are identical. For example, in FIG. 3 the edge 340 is a directed connection from the node 320 to the node 323, and the edge 342 is a directed connection from the node 330 to the node 332. An edge 340, . . . , 342 from a first node 320, . . . , 332 to a second node 320, . . . , 332 is also denoted as “ingoing edge” for the second node 320, . . . , 332 and as “outgoing edge” for the first node 320, . . . 332.

In this embodiment, the nodes 320, . . . , 332 of the artificial neural network 300 can be arranged in layers 310, . . . , 313, wherein the layers can comprise an intrinsic order introduced by the edges 340, . . . , 342 between the nodes 320, . . . , 332. In particular, edges 340, . . . , 342 can exist only between neighboring layers of nodes. In the displayed embodiment, there is an input layer 310 comprising only nodes 320, . . . 322 without an incoming edge, an output layer 313 comprising only nodes 331, 332 without outgoing edges, and hidden layers 311, 312 in-between the input layer 310 and the output layer 313. In general, the number of hidden layers 311, 312 can be chosen arbitrarily. The number of nodes 320, . . . , 322 within the input layer 310 usually relates to the number of input values of the neural network, and the number of nodes 331, 332 within the output layer 313 usually relates to the number of output values of the neural network.

In particular, a (real) number can be assigned as a value to every node 320, . . . , . . . , 332 of the neural network 300. Here, x⁽ⁿ⁾; denotes the value of the i-th node 320, . . . , 332 of the n-th layer 310, . . . , 313. The values of the nodes 320, . . . , 322 of the input layer 310 are equivalent to the input values of the neural network 300, the values of the nodes 331, 332 of the output layer 313 are equivalent to the output value of the neural network 300. Furthermore, each edge 340, . . . , 342 can comprise a weight being a real number, in particular, the weight is a real number within the interval [−1, 1] or within the interval [0, 1]. Here, w^{(m,n)}ᵢⱼ denotes the weight of the edge between the i-th node 320, . . . , 332 of the m-th layer 310, . . . , 313 and the j-th node 320, . . . , 332 of the n-th layer 310, . . . , 313. Furthermore, the abbreviation w (n) ij is defined for the weight w^{(m,n+1)}ᵢⱼ .

In particular, to calculate the output values of the neural network 300, the input values are propagated through the neural network. In particular, the values of the nodes 320, . . . , 332 of the (n+1)-th layer 310, . . . , 313 can be calculated based on the values of the nodes 320, . . . , 332 of the n-th layer 310, . . . , 313 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 310 are given by the input of the neural network 300, wherein values of the first hid-den layer 311 can be calculated based on the values of the input layer 310 of the neural network, wherein values of the second hidden layer 312 can be calculated based in the values of the first hidden layer 311, etc.

In order to set the values w^{(m,n)}ᵢⱼ for the edges, the neural network 300 has to be trained using training data. In particular, training data comprises training input data and training output data (denoted as tᵢ). For a training step, the neural network 300 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 300 (backpropagation algorithm). In particular, the weights are changed according to

$${w '}^{{( n )}_{i , j}} = {w}^{{( n )}_{i , j}} - y \cdot {δ}^{{( n )}_{j}} \cdot {x}^{{( n )}_{i}}$$

wherein y is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 313, wherein f′ is the first derivative of the activation function, and t⁽ⁿ⁺¹⁾ⱼ is the comparison training value for the j-th node of the output layer 313.

A convolutional neural network is a neural network that uses a convolution operation instead general matrix multiplication in at least one of its layers (so-called “convolutional layer”). In particular, a convolutional layer performs a dot product of one or more convolution kernels with the convolutional layer's input data/image, wherein the entries of the one or more convolution kernel are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

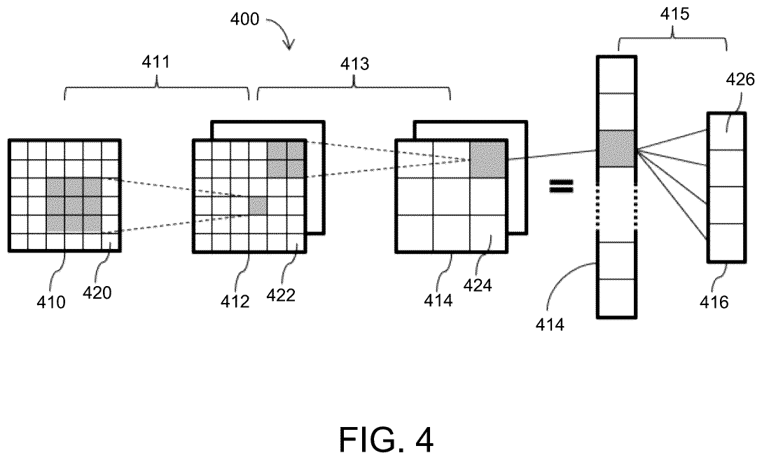

FIG. 4 shows an embodiment of a convolutional neural network 400 that may be used to implement one or more machine learning models described herein. In the displayed embodiment, the convolutional neural network comprises 400 an input node layer 410, a convolutional layer 411, a pooling layer 413, a fully connected layer 414 and an output node layer 416, as well as hidden node layers 412, 414. Alternatively, the convolutional neural network 400 can comprise several convolutional layers 411, several pooling layers 413 and several fully connected layers 415, as well as other types of layers. The order of the layers can be chosen arbitrarily, usually fully connected layers 415 are used as the last layers before the output layer 416.

In particular, within a convolutional neural network 400 nodes 420, 422, 424 of a node layer 410, 412, 414 can be considered to be arranged as a d-dimensional matrix or as a d-dimensional image. In particular, in the two-dimensional case the value of the node 420, 422, 424 indexed with i and j in the n-th node layer 410, 412, 414 can be denoted as x(n)[i, j]. However, the arrangement of the nodes 420, 422, 424 of one node layer 410, 412, 414 does not have an effect on the calculations executed within the convolutional neural network 400 as such, since these are given solely by the structure and the weights of the edges.

A convolutional layer 411 is a connection layer between an anterior node layer 410 (with node values x(n−1)) and a posterior node layer 412 (with node values x(n)). In particular, a convolutional layer 411 is characterized by the structure and the weights of the incoming edges forming a convolution operation based on a certain number of kernels. In particular, the structure and the weights of the edges of the convolutional layer 411 are chosen such that the values x (n) of the nodes 422 of the posterior node layer 412 are calculated as a convolution x(n)=K*x(n−1) based on the values x(n−1) of the nodes 420 anterior node layer 410, where the convolution * is defined in the two-dimensional case as

$${x}_{k}^{( n )} [ i , j ] = ( K * {x}^{( n - 1 )} ) [ i , j ] = {\sum }_{{i}^{'}} . {\sum }_{{j}^{'}} K [ {i}^{'} , {j}^{'} ] \cdot {x}^{( n - 1 )} [ i - {i}^{'} , j - {j}^{'} ] .$$

Here the kernel K is a d-dimensional matrix (in this embodiment, a two-dimensional matrix), which is usually small compared to the number of nodes 420, 422 (e.g., a 3×3 matrix, or a 5×5 matrix). In particular, this implies that the weights of the edges in the convolution layer 411 are not independent, but chosen such that they produce said convolution equation. In particular, for a kernel being a 3×3 matrix, there are only 9 independent weights (each entry of the kernel matrix corresponding to one independent weight), irrespectively of the number of nodes 420, 422 in the anterior node layer 410 and the posterior node layer 412.

In general, convolutional neural networks 400 use node layers 410, 412, 414 with a plurality of channels, in particular, due to the use of a plurality of kernels in convolutional layers 411. In those cases, the node layers can be considered as (d+1)-dimensional matrices (the first dimension indexing the channels). The action of a convolutional layer 411 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{'}} {\sum }_{{j}^{'}} {K}_{a , b} [ {i}^{'} , {j}^{'} ] \cdot {x}^{{( n - 1 )}_{a}} [ i - {i}^{'} , j - j ' ]$$

where x⁽ⁿ⁻¹⁾^{ₐ} corresponds to the a-th channel of the anterior node layer 410, x⁽ⁿ⁾^{_{b}} corresponds to the b-th channel of the posterior node layer 412 and K_{a,b} corresponds to one of the kernels. If a convolutional layer 411 acts on an anterior node layer 410 with A channels and outputs a posterior node layer 412 with B channels, there are A·B independent d-dimensional kernels K_{a,b}.

In general, in convolutional neural networks 400 activation functions are used. In this embodiment re ReLU (acronym for “Rectified Linear Units”) is used, with R(z)=max(0, z), so that the action of the convolutional layer 411 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 410 comprises 36 nodes 420, arranged as a two-dimensional 6×6 matrix. The first hidden node layer 412 comprises 72 nodes 422, arranged as two two-dimensional 6×6 matrices, each of the two matrices being the result of a convolution of the values of the input layer with a 3×3 kernel within the convolutional layer 411. Equivalently, the nodes 422 of the first hidden node layer 412 can be interpreted as arranged as a three-dimensional 2×6×6 matrix, wherein the first dimension correspond to the channel dimension.

The advantage of using convolutional layers 411 is that spatially local correlation of the input data can exploited by enforcing a local connectivity pattern between nodes of adjacent layers, in particular by each node being connected to only a small region of the nodes of the preceding layer.

A pooling layer 413 is a connection layer between an anterior node layer 412 (with node values x(n−1)) and a posterior node layer 414 (with node values x(n)). In particular, a pooling layer 413 can be characterized by the structure and the weights of the edges and the activation function forming a pooling operation based on a non-linear pooling function f. For example, in the two-dimensional case the values x(n) of the nodes 424 of the posterior node layer 414 can be calculated based on the values x(n−1) of the nodes 422 of the anterior node layer 412 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 413 the number of nodes 422, 424 can be reduced, by re-placing a number d1·d2 of neighboring nodes 422 in the anterior node layer 412 with a single node 422 in the posterior node layer 414 being calculated as a function of the values of said number of neighboring nodes. In particular, the pooling function f can be the max-function, the average or the L2-Norm. In particular, for a pooling layer 413 the weights of the incoming edges are fixed and are not modified by training.

The advantage of using a pooling layer 413 is that the number of nodes 422, 424 and the number of parameters is reduced. This leads to the amount of computation in the network being reduced and to a control of overfitting.

In the displayed embodiment, the pooling layer 413 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 400 are fully connected layers 415. A fully connected layer 415 is a connection layer between an anterior node layer 414 and a posterior node layer 416. A fully connected layer 413 can be characterized by the fact that a majority, in particular, all edges between nodes 414 of the anterior node layer 414 and the nodes 416 of the posterior node layer are present, and wherein the weight of each of these edges can be adjusted individually.

In this embodiment, the nodes 424 of the anterior node layer 414 of the fully connected layer 415 are displayed both as two-dimensional matrices, and additionally as non-related nodes (indicated as a line of nodes, wherein the number of nodes was reduced for a better presentability). This operation is also denoted as “flattening”. In this embodiment, the number of nodes 426 in the posterior node layer 416 of the fully connected layer 415 smaller than the number of nodes 424 in the anterior node layer 414. Alternatively, the number of nodes 426 can be equal or larger.

Furthermore, in this embodiment the Softmax activation function is used within the fully connected layer 415. By applying the Softmax function, the sum the values of all nodes 426 of the output layer 416 is 1, and all values of all nodes 426 of the output layer 416 are real numbers between 0 and 1. In particular, if using the convolutional neural network 400 for categorizing input data, the values of the output layer 416 can be interpreted as the probability of the input data falling into one of the different categories.

In particular, convolutional neural networks 400 can be trained based on the backpropagation algorithm. For preventing overfitting, methods of regularization can be used, e.g., dropout of nodes 420, . . . , 424, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

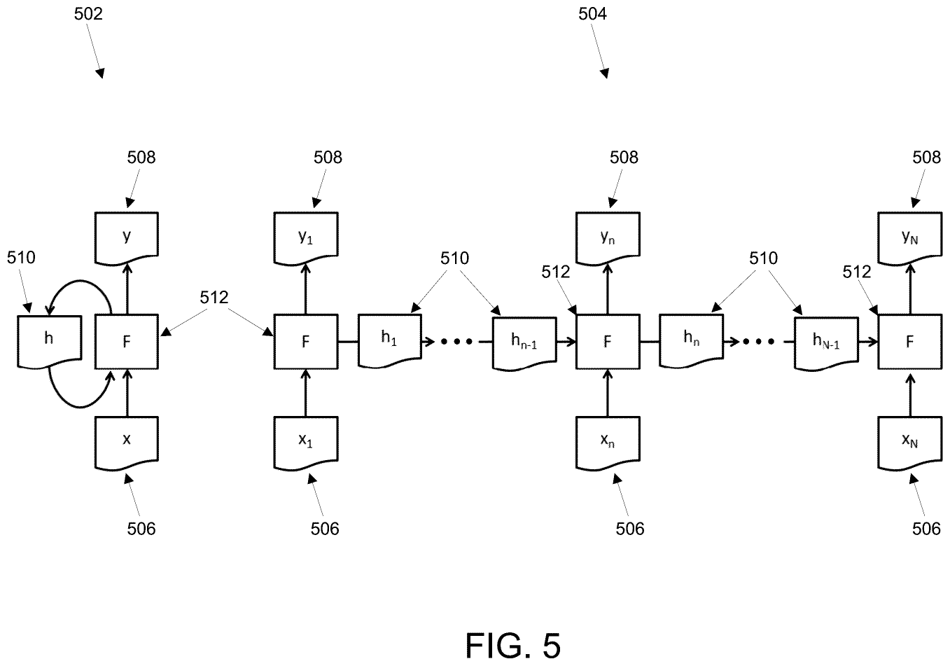

FIG. 5 shows the schematic structure of a recurrent machine learning model F, both in a recurrent representation 502 and in an unfolded representation 504, that may be used to implement one or more machine learning models described herein. The recurrent machine learning model takes as input several input datasets x, x₁, . . . , X_{N} 506 and creates a corresponding set of output datasets y, y₁, . . . , y_{N} 508. Furthermore, the output depends on a so-called hidden vector h, h₁, . . . , h_{N} 510, which implicitly comprises information about input datasets previously used as input for the recurrent machine learning model F 512. By using these hidden vectors h, h₁, . . . , h_{N} 510, a sequentiality of the input datasets can be leveraged.

In a single step of the processing, the recurrent machine learning model F 512 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=Fxₙ, hₙ₋₁), or by splitting the recurrent machine learning model F 512 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, ho can be chosen randomly or filled with all entries being zero. The parameters of the recurrent machine learning model F 512 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(Xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 602 that may be used to implement systems, apparatuses, and methods described herein is depicted in FIG. 6. Computer 602 includes a processor 604 operatively coupled to a data storage device 612 and a memory 610. Processor 604 controls the overall operation of computer 602 by executing computer program instructions that define such operations. The computer program instructions may be stored in data storage device 612, or other computer readable medium, and loaded into memory 610 when execution of the computer program instructions is desired. Thus, the method and workflow steps or functions of FIG. 1 or 2 can be defined by the computer program instructions stored in memory 610 and/or data storage device 612 and controlled by processor 604 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 604 executes the method and workflow steps or functions of FIG. 1 or 2. Computer 602 may also include one or more network interfaces 606 for communicating with other devices via a network. Computer 602 may also include one or more input/output devices 608 that enable user interaction with computer 602 (e.g., display, keyboard, mouse, speakers, buttons, etc.).

Processor 604 may include both general and special purpose microprocessors, and may be the sole processor or one of multiple processors of computer 602. Processor 604 may include one or more central processing units (CPUs), for example. Processor 604, data storage device 612, and/or memory 610 may include, be supplemented by, or incorporated in, one or more application-specific integrated circuits (ASICs) and/or one or more field programmable gate arrays (FPGAs).

Data storage device 612 and memory 610 each include a tangible non-transitory computer readable storage medium. Data storage device 612, and memory 610, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 608 may include peripherals, such as a printer, scanner, display screen, etc. For example, input/output devices 608 may include a display device such as a cathode ray tube (CRT) or liquid crystal display (LCD) monitor for displaying information to the user, a keyboard, and a pointing device such as a mouse or a trackball by which the user can provide input to computer 602.

An image acquisition device 614 can be connected to the computer 602 to input image data (e.g., medical images) to the computer 602. It is possible to implement the image acquisition device 614 and the computer 602 as one device. It is also possible that the image acquisition device 614 and the computer 602 communicate wirelessly through a network. In a possible embodiment, the computer 602 can be located remotely with respect to the image acquisition device 614.

Any or all of the systems, apparatuses, and methods discussed herein may be implemented using one or more computers such as computer 602.

One skilled in the art will recognize that an implementation of an actual computer or computer system may have other structures and may contain other components as well, and that FIG. 6 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving 1) medical imaging metadata in a first format and 2) instructions; converting the medical imaging metadata from the first format to a second format based on the instructions using a machine learning based model; and outputting the medical imaging metadata in the second format.

Illustrative embodiment 2. The computer-implemented method according to illustrative embodiment 1, wherein the machine learning based model is an LLM (large language model) and receiving 1) medical imaging metadata in a first format and 2) instructions comprises: receiving one or more prompts comprising 1) the medical imaging metadata in the first format and 2) the instructions.

Illustrative embodiment 3. The computer-implemented method according to any one of illustrative embodiments 1-2, wherein the machine learning based model receives as input the medical imaging metadata in the first format and the instructions and generates as output the medical imaging metadata in the second format.

Illustrative embodiment 4. The computer-implemented method according to any one of illustrative embodiments 1-3, wherein the medical imaging metadata is metadata associated with an acquisition of one or more medical images of a patient.

Illustrative embodiment 5. The computer-implemented method according to illustrative embodiment 4, wherein the medical imaging metadata comprises patient health information of the patient and image acquisition parameters of the one or more medical images.

Illustrative embodiment 6. The computer-implemented method according to any one of illustrative embodiments 1-5, wherein the first format and the second format are different implementations of a DICOM (digital imaging and communications in medicine) format.

Illustrative embodiment 7. The computer-implemented method according to any one of illustrative embodiments 1-6, wherein the instructions comprise instructions for converting the medical imaging metadata from the first format to the second format.

Illustrative embodiment 8. The computer-implemented method according to any one of illustrative embodiments 1-7, wherein converting the medical imaging metadata from the first format to a second format based on the instructions using a machine learning based model comprises: converting the medical imaging metadata from the first format to the second format in response to determining that the medical imaging metadata in the second format includes or is missing information for a class of patients that would influence a future processing step.

Illustrative embodiment 9. The computer-implemented method according to any one of illustrative embodiments 1-8, further comprising: extracting information from a medical image associated with the medical imaging metadata using a machine learning based image assessment model; and comparing the extracted information with the medical imaging metadata to confirm accuracy of the medical imaging metadata.

Illustrative embodiment 10. An apparatus comprising: means for receiving 1) medical imaging metadata in a first format and 2) instructions; means for converting the medical imaging metadata from the first format to a second format based on the instructions using a machine learning based model; and means for outputting the medical imaging metadata in the second format.

Illustrative embodiment 11. The apparatus according to illustrative embodiment 10, wherein the machine learning based model is an LLM (large language model) and the means for receiving 1) medical imaging metadata in a first format and 2) instructions comprises: means for receiving one or more prompts comprising 1) the medical imaging metadata in the first format and 2) the instructions.

Illustrative embodiment 12. The apparatus according to any one of illustrative embodiments 10-11, wherein the machine learning based model receives as input the medical imaging metadata in the first format and the instructions and generates as output the medical imaging metadata in the second format.

Illustrative embodiment 13. The apparatus according to any one of illustrative embodiments 10-12, wherein the medical imaging metadata is metadata associated with an acquisition of one or more medical images of a patient.

Illustrative embodiment 14. The apparatus according to illustrative embodiment 10-13, wherein the medical imaging metadata comprises patient health information of the patient and image acquisition parameters of the one or more medical images.

Illustrative embodiment 15. A non-transitory computer-readable storage medium comprising instructions which, when executed by a computer, cause the computer to carry out operations comprising: receiving 1) medical imaging metadata in a first format and 2) instructions; converting the medical imaging metadata from the first format to a second format based on the instructions using a machine learning based model; and outputting the medical imaging metadata in the second format.

Illustrative embodiment 16. The non-transitory computer-readable storage medium according to illustrative embodiment 15, wherein the machine learning based model is an LLM (large language model) and receiving 1) medical imaging metadata in a first format and 2) instructions comprises: receiving one or more prompts comprising 1) the medical imaging metadata in the first format and 2) the instructions.

Illustrative embodiment 17. The non-transitory computer-readable storage medium according to any one of illustrative embodiments 15-16, wherein the first format and the second format are different implementations of a DICOM (digital imaging and communications in medicine) format.

Illustrative embodiment 18. The non-transitory computer-readable storage medium according to any one of illustrative embodiments 15-17, wherein the instructions comprise instructions for converting the medical imaging metadata from the first format to the second format.

Illustrative embodiment 19. The non-transitory computer-readable storage medium according to any one of illustrative embodiments 15-18, wherein converting the medical imaging metadata from the first format to a second format based on the instructions using a machine learning based model comprises: converting the medical imaging metadata from the first format to the second format in response to determining that the medical imaging metadata in the second format includes or is missing information for a class of patients that would influence a future processing step.

Illustrative embodiment 20. The non-transitory computer-readable storage medium according to any one of illustrative embodiments 15-19, the operations further comprising: extracting information from a medical image associated with the medical imaging metadata using a machine learning based image assessment model; and comparing the extracted information with the medical imaging metadata to confirm accuracy of the medical imaging metadata.

1. A computer-implemented method comprising:
receiving 1) medical imaging metadata in a first format and 2) instructions;
converting the medical imaging metadata from the first format to a second format based on the instructions using a machine learning based model; and
outputting the medical imaging metadata in the second format.

2. The computer-implemented method of claim 1, wherein the machine learning based model is an LLM (large language model) and receiving 1) medical imaging metadata in a first format and 2) instructions comprises:
receiving one or more prompts comprising 1) the medical imaging metadata in the first format and 2) the instructions.

3. The computer-implemented method of claim 1, wherein the machine learning based model receives as input the medical imaging metadata in the first format and the instructions and generates as output the medical imaging metadata in the second format.

4. The computer-implemented method of claim 1, wherein the medical imaging metadata is metadata associated with an acquisition of one or more medical images of a patient.

5. The computer-implemented method of claim 4, wherein the medical imaging metadata comprises patient health information of the patient and image acquisition parameters of the one or more medical images.

6. The computer-implemented method of claim 1, wherein the first format and the second format are different implementations of a DICOM (digital imaging and communications in medicine) format.

7. The computer-implemented method of claim 1, wherein the instructions comprise instructions for converting the medical imaging metadata from the first format to the second format.

8. The computer-implemented method of claim 1, wherein converting the medical imaging metadata from the first format to a second format based on the instructions using a machine learning based model comprises:
converting the medical imaging metadata from the first format to the second format in response to determining that the medical imaging metadata in the second format includes or is missing information for a class of patients that would influence a future processing step.

9. The computer-implemented method of claim 1, further comprising:
extracting information from a medical image associated with the medical imaging metadata using a machine learning based image assessment model; and
comparing the extracted information with the medical imaging metadata to confirm accuracy of the medical imaging metadata.

10. An apparatus comprising:
means for receiving 1) medical imaging metadata in a first format and 2) instructions;
means for converting the medical imaging metadata from the first format to a second format based on the instructions using a machine learning based model; and
means for outputting the medical imaging metadata in the second format.

11. The apparatus of claim 10, wherein the machine learning based model is an LLM (large language model) and the means for receiving 1) medical imaging metadata in a first format and 2) instructions comprises:
means for receiving one or more prompts comprising 1) the medical imaging metadata in the first format and 2) the instructions.

12. The apparatus of claim 10, wherein the machine learning based model receives as input the medical imaging metadata in the first format and the instructions and generates as output the medical imaging metadata in the second format.

13. The apparatus of claim 10, wherein the medical imaging metadata is metadata associated with an acquisition of one or more medical images of a patient.

14. The apparatus of claim 13, wherein the medical imaging metadata comprises patient health information of the patient and image acquisition parameters of the one or more medical images.

15. A non-transitory computer-readable storage medium comprising instructions which, when executed by a computer, cause the computer to carry out operations comprising:
receiving 1) medical imaging metadata in a first format and 2) instructions;
converting the medical imaging metadata from the first format to a second format based on the instructions using a machine learning based model; and
outputting the medical imaging metadata in the second format.

16. The non-transitory computer-readable storage medium of claim 15, wherein the machine learning based model is an LLM (large language model) and receiving 1) medical imaging metadata in a first format and 2) instructions comprises:
receiving one or more prompts comprising 1) the medical imaging metadata in the first format and 2) the instructions.

17. The non-transitory computer-readable storage medium of claim 15, wherein the first format and the second format are different implementations of a DICOM (digital imaging and communications in medicine) format.

18. The non-transitory computer-readable storage medium of claim 15, wherein the instructions comprise instructions for converting the medical imaging metadata from the first format to the second format.

19. The non-transitory computer-readable storage medium of claim 15, wherein converting the medical imaging metadata from the first format to a second format based on the instructions using a machine learning based model comprises:
converting the medical imaging metadata from the first format to the second format in response to determining that the medical imaging metadata in the second format includes or is missing information for a class of patients that would influence a future processing step.

20. The non-transitory computer-readable storage medium of claim 15, the operations further comprising:
extracting information from a medical image associated with the medical imaging metadata using a machine learning based image assessment model; and
comparing the extracted information with the medical imaging metadata to confirm accuracy of the medical imaging metadata.
